(19) United States
(12) Patent Application Publication
(10) Pub. No.: US 2012/0309792 A1
(43) Pub. Date: Dec. 6, 2012

(54) TETRAZOLE DERIVATIVES AS NICOTINIC ACETYLCHOLINE RECEPTOR MODULATORS

(75) Inventors: Dan Peters, Malmo (SE); Morten Grunnet, Copenhagen O (DK); Daniel B. Timmermann, Herlev (DK); Birgitte L. Eriksen, Farum (DK); Tino Dyhring, Solrod (DK); Philip K. Ahring, Bagsvaerd (DK)

(73) Assignee: NEUROSEARCH A/S, Ballerup (DK)

(21) Appl. No.: 13/516,279

(22) PCT Filed: Dec. 16, 2010
(86) PCT No.: PCT/EP10/69853

§ 371 Date: Jul. 11, 2012

Related U.S. Application Data
(60) Provisional application No. 61/289,805, filed on Dec. 23, 2009.

(30) Foreign Application Priority Data
Dec. 18, 2009 (DK) PA 2009 70280

(51) Int. Cl. A61K 31/444; A61P 25/00; A61P 25/18; A61P 25/24; A61P 25/22; A61P 3/04; A61P 25/28; A61P 25/16; A61P 25/08; A61P 7/06; A61P 25/14; A61P 25/06; A61P 9/06; A61P 21/00; A61P 9/10; A61P 15/06; A61P 11/06; A61P 1/12; A61P 15/00; A61P 15/10; A61P 9/12; A61P 29/00; A61P 17/10; A61P 17/00; A61P 1/00; A61P 1/04; A61P 25/30; A61P 25/34; A61P 25/36; A61P 25/32; C07D 401/14

(52) U.S. Cl. 514/333; 546/256

(57) ABSTRACT

This invention relates to novel tetrazole derivatives, which are found to be modulators of the nicotinic acetylcholine receptors. Due to their pharmacological profile the compounds of the invention may be useful for the treatment of diseases or disorders as diverse as those related to the cholinergic system of the central nervous system (CNS), the peripheral nervous system (PNS), diseases or disorders related to smooth muscle contraction, endocrine diseases or disorders, diseases or disorders related to neuro-degeneration, diseases or disorders related to inflammation, pain, and withdrawal symptoms caused by the termination of abuse of chemical substances.

### TECHNICAL FIELD

This invention relates to novel tetrazole derivatives, which are found to be modulators of the nicotinic acetylcholine receptors. Due to their pharmacological profile the compounds of the invention may be useful for the treatment of diseases or disorders as diverse as those related to the cholinergic system of the central nervous system (CNS), the peripheral nervous system (PNS), diseases or disorders related to smooth muscle contraction, endocrine diseases or disorders, diseases or disorders related to neuro-degeneration, diseases or disorders related to inflammation, pain, and withdrawal symptoms caused by the termination of abuse of chemical substances.

### BACKGROUND ART

The endogenous cholinergic neurotransmitter, acetylcholine, exerts its biological effect via two types of cholinergic receptors, the muscarinic Acetyl Choline Receptors (mAChR) and the nicotinic Acetyl Choline Receptors (nAChR).

Nicotinic acetylcholine receptors (nAChRs) are pentameric ligand gated ion channels and widely distributed throughout the central (CNS) and peripheral (PNS) nervous systems. At least 12 subunit proteins, i.e. α2-α10 and β2-β4, have been identified in neuronal tissue. These subunits provide for a great variety of homomeric and heteromeric combinations that account for the diverse receptor subtypes. For example, the predominant receptor that is responsible for high affinity binding of nicotine in brain tissue has composition α4β2, while another major population of receptors is comprised of the homomeric α7.

Discovery of the important role played by nAChRs in several CNS disorders has called attention to these membrane proteins and to ligands able to modulate their functions. The existence of different subtypes at multiple levels has complicated the understanding of this receptor's physiological role, but at the same time has increased the efforts to discover selective compounds in order to improve the pharmacological characterization of this kind of receptor and to make safer the possible therapeutic use of its modulators.

Shawali A S and Fahmi A A [Journal of Heterocyclic Chemistry (1979), 16, (1), 123-8] describe azo coupling of benzenesulfonylhydrazones of heterocyclic aldehydes.

WO 03/029210 describes heteroaryl substituted tetrazole modulators of metabotropic glutamate receptor-5.

### SUMMARY OF THE INVENTION

The present invention is devoted to the provision modulators of the nicotinic receptors, which modulators are useful for the treatment of diseases or disorders related to the nicotinic acetylcholine receptor (nAChR).

Due to their pharmacological profile the compounds of the invention may be useful for the treatment of diseases or disorders as diverse as those related to the cholinergic system of the central nervous system (CNS), the peripheral nervous system (PNS), diseases or disorders related to smooth muscle contraction, endocrine diseases or disorders, diseases or disorders related to neuro-degeneration, diseases or disorders related to inflammation, pain, and abuse liability and withdrawal symptoms caused by the termination of abuse of chemical substances, in particular nicotine.

The compounds of the invention may also be useful as diagnostic tools or monitoring agents in various diagnostic methods, and in particular for in vivo receptor imaging (neuroimaging), and they may be used in labelled or unlabelled form.

In its first aspect the invention provides a tetrazole derivative represented by Formula I

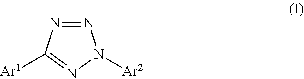

a stereoisomer thereof or a mixture of its stereoisomers, or a pharmaceutically acceptable addition salt thereof, wherein both of Ar¹ and Ar² represent pyridinyl, optionally substituted one or two times with substituents selected from alkyl, alkoxy and halo.

In a second aspect the invention provides pharmaceutical compositions comprising a therapeutically effective amount of the tetrazole derivative of the invention, or a pharmaceutically acceptable addition salt thereof, together with at least one pharmaceutically acceptable carrier or diluent.

Viewed from another aspect the invention relates to the use of the tetrazole derivative of the invention, or a pharmaceutically acceptable addition salt thereof, for the manufacture of pharmaceutical compositions/medicaments for the treatment, prevention or alleviation of a disease or a disorder or a condition of a mammal, including a human, which disease, disorder or condition is responsive to modulation of cholinergic receptors.

In yet another aspect the invention provides a method for treatment, prevention or alleviation of diseases, disorders or conditions of a living animal body, including a human, which disorder, disease or condition is responsive to modulation of cholinergic receptors, and which method comprises the step of administering to such a living animal body in need thereof a therapeutically effective amount of the tetrazole derivative of the invention.

Other objects of the invention will be apparent to the person skilled in the art from the following detailed description and examples.

### DETAILED DISCLOSURE OF THE INVENTION

### Tetrazole Derivatives

In its first aspect the invention provides a tetrazole derivative represented by Formula I

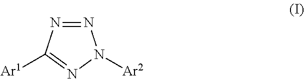

a stereoisomer thereof or a mixture of its stereoisomers, or a pharmaceutically acceptable addition salt thereof, wherein both of Ar¹ and Ar² represent pyridinyl, optionally substituted one or two times with substituents selected from alkyl, alkoxy and halo.

In a more preferred embodiment both of Ar¹ and Ar² represent pyridinyl, and in particular 3-pyridinyl, optionally substituted with alkyl, alkoxy or halo.

In another more preferred embodiment both of Ar¹ and Ar² represent pyridinyl, and in particular 3-pyridinyl, optionally substituted with alkyl, and in particular methyl.

In a third more preferred embodiment both of Ar¹ and Ar² represent pyridinyl, and in particular 3-pyridinyl, optionally substituted with alkoxy, and in particular methoxy.

In a fourth more preferred embodiment both of Ar¹ and Ar² represent pyridinyl, and in particular 3-pyridinyl, optionally substituted with halo, and in particular fluoro, chloro or bromo.

In a fifth more preferred embodiment both of Ar¹ and Ar² represent pyridinyl, and in particular 3-pyridinyl.

In a preferred embodiment the tetrazole derivative of the invention is a compound of Formula I, a stereoisomer thereof or a mixture of its stereoisomers, or a pharmaceutically acceptable addition salt thereof, wherein Ar¹ represents pyridinyl, and in particular 3-pyridinyl, optionally substituted with alkyl or halo; and Ar² represents pyridinyl, and in particular 3-pyridinyl, optionally substituted with alkyl, alkoxy or halo.

In a further embodiment the tetrazole derivative of the invention is a compound of Formula I, a stereoisomer thereof or a mixture of its stereoisomers, or a pharmaceutically acceptable addition salt thereof, wherein Ar¹ represents 3-pyridinyl, optionally substituted with alkyl or halo; and Ar² represents 3-pyridinyl, optionally substituted with alkyl, alkoxy or halo; with the proviso that at least one of Ar¹ and Ar² represents a substituted 3-pyridinyl. In a still further embodiment the tetrazole derivative of the invention is a compound of Formula I, a stereoisomer thereof or a mixture of its stereoisomers, or a pharmaceutically acceptable addition salt thereof, wherein Ar¹ represents 3-pyridinyl, optionally substituted with alkyl or halo; and Ar² represents 3-pyridinyl, optionally substituted with alkyl, alkoxy or halo; with the proviso that at least one of Ar¹ and Ar² represents a substituted 3-pyridinyl; and with the further proviso that Ar² does not represent 6-chloro-pyridin-3-yl if Ar¹ represents pyridin-3-yl or 2-chloro-pyridin-3-yl.

In a more preferred embodiment Ar¹ represents pyridinyl, and in particular 3-pyridinyl; and Ar² represents pyridinyl, and in particular 3-pyridinyl, optionally substituted with alkyl, alkoxy or halo.

In another more preferred embodiment Ar¹ represents pyridinyl, and in particular 3-pyridinyl; and Ar² represents pyridinyl, and in particular 3-pyridinyl, substituted with alkyl, alkoxy or halo.

In a third more preferred embodiment Ar¹ represents pyridinyl, and in particular 3-pyridinyl; and Ar² represents pyridinyl, and in particular 3-pyridinyl, substituted with alkyl, and in particular methyl. In a special embodiment Ar² represents 2-methyl-pyridin-3-yl, 5-methyl-pyridin-3-yl or 6-methyl-pyridin-3-yl.

In a fourth more preferred embodiment Ar¹ represents pyridinyl, and in particular 3-pyridinyl; and Ar² represents pyridinyl, and in particular 3-pyridinyl, substituted with alkoxy, and in particular methoxy. In a special embodiment Ar² represents 5-methoxy-pyridin-3-yl.

In a fifth more preferred embodiment Ar¹ represents pyridinyl, and in particular 3-pyridinyl; and Ar² represents pyridinyl, and in particular 3-pyridinyl, substituted with halo, and in particular chloro, fluoro or bromo, such as fluoro or bromo. In a special embodiment Ar² represents 5-bromo-pyridin-3-yl, 5-chloro-pyridin-3-yl, 5-fluoro-pyridin-3-yl, 6-chloro-pyridin-3-yl or 6-fluoro-pyridin-3-yl.

In a sixth more preferred embodiment Ar¹ represents 3-pyridinyl, substituted with alkyl or halo; and Ar² represents 3-pyridinyl, or 3-pyridinyl substituted with halo, and in particular fluoro.

In a seventh more preferred embodiment Ar¹ represents 3-pyridinyl substituted with alkyl, and in particular methyl; and Ar² represents 3-pyridinyl, or 3-pyridinyl substituted with halo, and in particular fluoro. In a special embodiment, Ar¹ represents 2-methyl-pyridin-3-yl or 6-methyl-pyridin-3-yl and Ar² represents pyridine-3-yl.

In an eight more preferred embodiment Ar¹ represents 3-pyridinyl substituted with halo, and in particular fluoro or chloro; and Ar² represents 3-pyridinyl, or 3-pyridinyl substituted with halo, and in particular fluoro. In a special embodiment, Ar¹ represents 2-fluoro-pyridin-3-yl, 5-fluoro-pyridin-3-yl or 6-fluoro-pyridin-3-yl and Ar² represents pyridin-3-yl, 5-fluoro-pyridin-3-yl, 6-chloro-pyridin-3-yl or 6-fluoro-pyridin-3-yl. In a further special embodiment, Ar¹ represents 5-chloro-pyridin-3-yl or 6-chloro-pyridin-3-yl and Ar² represents pyridin-3-yl, 5-fluoro-pyridin-3-yl, 6-chloro-pyridin-3-yl or 6-fluoro-pyridin-3-yl. In a still further special embodiment, Ar¹ represents 5-bromo-pyridin-3-yl or 5-iodo-pyridin-3-yl and Ar² represents pyridin-3-yl or 6-fluoro-pyridin-3-yl.

In a most preferred embodiment the tetrazole derivative of the invention is
3-Fluoro-5-[5-(3-pyridyl)tetrazol-2-yl]pyridine;
3-[2-(3-Pyridyl)tetrazol-5-yl]pyridine fumarate;
3-Bromo-5-[5-(3-pyridyl)tetrazol-2-yl]pyridine;
3-Methoxy-5-[5-(3-pyridyl)tetrazol-2-yl]pyridine;
2-Methyl-5-[2-(3-pyridyl)tetrazol-5-yl]pyridine;
2-Methyl-5-[5-(3-pyridyl)tetrazol-2-yl]pyridine;
3-Methyl-5-[5-(3-pyridyl)tetrazol-2-yl]pyridine;
2-Methyl-3-[5-(3-pyridyl)tetrazol-2-yl]pyridine;
2-Methyl-3-[2-(3-pyridyl)tetrazol-5-yl]pyridine;
2-Fluoro-5-[5-(3-pyridyl)tetrazol-2-yl]pyridine;
3-Fluoro-5-[2-(5-fluoro-3-pyridyl)tetrazol-5-yl]pyridine;
3-Fluoro-5-[2-(6-fluoro-3-pyridyl)tetrazol-5-yl]pyridine;
2-Fluoro-5-[2-(5-fluoro-3-pyridyl)tetrazol-5-yl]pyridine;
2-Chloro-5-[2-(3-pyridyl)tetrazol-5-yl]pyridine;
2-Fluoro-5-[2-(3-pyridyl)tetrazol-5-yl]pyridine;
3-Chloro-5-[2-(3-pyridyl)tetrazol-5-yl]pyridine;
3-Fluoro-5-[2-(3-pyridyl)tetrazol-5-yl]pyridine;
2-Chloro-5-[5-(3-pyridyl)tetrazol-2-yl]pyridine;
3-Chloro-5-[2-(6-fluoro-3-pyridyl)tetrazol-5-yl]pyridine;
2-Chloro-5-[5-(5-fluoro-3-pyridyl)tetrazol-2-yl]pyridine;
2-Chloro-5-[2-(5-fluoro-3-pyridyl)tetrazol-5-yl]pyridine;
3-Chloro-5-[5-(3-pyridyl)tetrazol-2-yl]pyridine;
3-Chloro-5-[2-(5-fluoro-3-pyridyl)tetrazol-5-yl]pyridine;
3-Bromo-5-[2-(3-pyridyl)tetrazol-5-yl]pyridine;
2-Fluoro-3-[2-(6-fluoro-3-pyridyl)tetrazol-5-yl]pyridine;
2-Fluoro-5-[5-(5-iodo-3-pyridyl)tetrazol-2-yl]pyridine; or
2-Chloro-5-[5-(5-chloro-3-pyridyl)tetrazol-2-yl]pyridine;

a stereoisomer thereof or a mixture of its stereoisomers, or a

pharmaceutically acceptable addition salt thereof.

Any combination of two or more of the embodiments described herein is considered within the scope of the present invention.

### Definition of Substituents

In the context of this invention halo represents fluoro, chloro, bromo or iodo.

In the context of this invention an alkyl group designates a univalent saturated, straight or branched hydrocarbon chain. The hydrocarbon chain preferably contain of from one to eighteen carbon atoms (C₁₋₁₈-alkyl), more preferred of from one to six carbon atoms (C₁₋₈-alkyl; lower alkyl), including pentyl, isopentyl, neopentyl, hexyl and isohexyl. In a preferred embodiment alkyl represents a C₁₋₄-alkyl group, including butyl, isobutyl, secondary butyl, and tertiary butyl. In another preferred embodiment of this invention alkyl represents a C₁₋₃-alkyl group, which may in particular be methyl, ethyl, propyl or isopropyl.

In the context of this invention an alkoxy group designates an “alkyl-O—” group, wherein alkyl is as defined above. Examples of preferred alkoxy groups of the invention include methoxy, ethoxy and isopropoxy.

### Pharmaceutically Acceptable Salts

The tetrazole derivative of the invention may be provided in any form suitable for the intended administration. Suitable forms include pharmaceutically (i.e. physiologically) acceptable salts, and pre- or prodrug forms of the compound of the invention.

Examples of pharmaceutically acceptable addition salts include, without limitation, the non-toxic inorganic and organic acid addition salts such as the hydrochloride, the hydrobromide, the nitrate, the perchlorate, the phosphate, the sulphate, the formate, the acetate, the aconate, the ascorbate, the benzene-sulphonate, the benzoate, the cinnamate, the citrate, the embonate, the enantate, the fumarate, the glutamate, the glycolate, the lactate, the maleate, the malonate, the mandelate, the methanesulphonate, the naphthalene-2-sulphonate derived, the phthalate, the salicylate, the sorbate, the stearate, the succinate, the tartrate, the toluene-p-sulphonate, and the like. Such salts may be formed by procedures well known and described in the art.

Other acids such as oxalic acid, which may not be considered pharmaceutically acceptable, may be useful in the preparation of salts useful as intermediates in obtaining a chemical compound of the invention and its pharmaceutically acceptable acid addition salt.

Metal salts of a compound of the invention include alkali metal salts, such as the sodium salt of a compound of the invention containing a carboxy group.

Examples of pharmaceutically acceptable cationic salts of a chemical compound of the invention include, without limitation, the sodium, the potassium, the calcium, the magnesium, the zinc, the aluminium, the lithium, the choline, the lysinium, and the ammonium salt, and the like, of a chemical compound of the invention containing an anionic group. Such cationic salts may be formed by procedures well known and described in the art.

The chemical compound of the invention may be provided in dissoluble or indissoluble forms together with a pharmaceutically acceptable solvent such as water, ethanol, and the like. Dissoluble forms may also include hydrated forms such as the monohydrate, the dihydrate, the hemihydrate, the trihydrate, the tetrahydrate, and the like. In general, the dissoluble forms are considered equivalent to indissoluble forms for the purposes of this invention.

In the context of this invention the “onium salts” of N-containing compounds may also be contemplated as pharmaceutically acceptable salts. Preferred “onium salts” include the alkyl-onium salts, the cycloalkyl-onium salts, and the cycloalkylalkyl-onium salts.

In the context of this invention the “onium salts” of N-containing compounds may also be contemplated as pharmaceutically acceptable salts. Preferred “onium salts” include the alkyl-onium salts, the cycloalkyl-onium salts, and the cycloalkylalkyl-onium salts.

### Steric Isomers

It will be appreciated by those skilled in the art that the compounds of the present invention may exist in different stereoisomeric forms, including enantiomers, diastereomers, as well as geometric isomers (cis-trans isomers). The invention includes all such stereoisomers and any mixtures thereof including racemic mixtures.

Racemic forms can be resolved into the optical antipodes by known methods and techniques. One way of separating the enantiomeric compounds (including enantiomeric intermediates) is by use of an optically active amine, and liberating the diastereomeric, resolved salt by treatment with an acid. Another method for resolving racemates into the optical antipodes is based upon chromatography on an optical active matrix. Racemic compounds of the present invention can thus be resolved into their optical antipodes, e.g., by fractional crystallisation of D- or L- (tartrates, mandelates or camphorsulphonate) salts for example.

Additional methods for the resolving the optical isomers are known in the art. Such methods include those described by Jaques J, Collet A, & Wilen S in “Enantiomers, Racemates, and Resolutions”, John Wiley and Sons, New York (1981).

Optical active compounds can also be prepared from optical active starting materials or intermediates.

### Labelled Compounds

The compounds of the invention may be used in their labelled or unlabelled form. In the context of this invention the labelled compound has one or more atoms replaced by an atom having an atomic mass or mass number different from the atomic mass or mass number usually found in nature. The labelling will allow easy quantitative detection of said compound.

The labelled compounds of the invention may be useful as diagnostic tools, radio tracers, or monitoring agents in various diagnostic methods, and for in vivo receptor imaging.

The labelled isomer of the invention preferably contains at least one radionuclide as a label. Positron emitting radionuclides are all candidates for usage. In the context of this invention the radionuclide is preferably selected from ²H (deuterium), ³H (tritium), ¹¹C, ¹³C, ¹⁴C, ¹³¹I, ¹²⁵I, ¹²³I, and ¹⁸F.

The physical method for detecting the labelled isomer of the present invention may be selected from Position Emission Tomography (PET), Single Photon Imaging Computed Tomography (SPECT), Magnetic Resonance Spectroscopy (MRS), Magnetic Resonance Imaging (MRI), and Computed Axial X-ray Tomography (CAT), or combinations thereof.

### Methods of Producing Tetrazole Derivatives

The tetrazole derivative of the invention may be prepared by conventional methods for chemical synthesis, e.g. those described in the working examples. The starting materials for the processes described in the present application are known or may readily be prepared by conventional methods from commercially available chemicals.

Also one compound of the invention can be converted to another compound of the invention using conventional methods.

The end products of the reactions described herein may be isolated by conventional techniques, e.g. by extraction, crystallisation, distillation, chromatography, etc.

### Biological Activity

The present invention is devoted to the provision modulators of the nicotinic receptors, which modulators are useful for the treatment of diseases or disorders related to the nicotinic acetylcholine receptor (nAChR). Preferred compounds of the invention show a positive allosteric modulation of the nicotinic acetylcholine α4β2 receptor subtypes.

Due to their pharmacological profile the compounds of the invention may be useful for the treatment of diseases or disorders as diverse as those related to the cholinergic system of the central nervous system (CNS), the peripheral nervous system (PNS), diseases or disorders related to smooth muscle contraction, endocrine diseases or disorders, diseases or disorders related to neuro-degeneration, diseases or disorders related to inflammation, pain, and abuse liability and withdrawal symptoms caused by the termination of abuse of chemical substances, in particular nicotine.

In a preferred embodiment the disease, disorder or condition relates to the central nervous system.

The compounds of the invention may also be useful as diagnostic tools or monitoring agents in various diagnostic methods, and in particular for in vivo receptor imaging (neuroimaging), and they may be used in labelled or unlabelled form.

In another preferred embodiment the disease, disorder or condition is a cognitive disorder, learning deficit, memory deficits and dysfunction, Down's syndrome, Alzheimer's disease, attention deficit, attention deficit hyperactivity disorder (ADHD), Tourette's syndrome, psychosis, depression, bipolar disorder, mania, manic depression, schizophrenia, cognitive or attention deficits related to schizophrenia, obsessive compulsive disorders (OCD), panic disorders, eating disorders, in particular anorexia nervosa, bulimia and obesity, narcolepsy, nociception, AIDS-dementia, senile dementia, autism, Parkinson's disease, Huntington's disease, amyotrophic lateral sclerosis (ALS), anxiety, non-OCD anxiety disorders, convulsive disorders, convulsions, epilepsy, neurodegenerative disorders, transient anoxia, induced neuro-degeneration, neuropathy, diabetic neuropathy, peripheral dyslexia, tardive dyskinesia, hyperkinesia, pain, mild pain, moderate or severe pain, pain of acute, chronic or recurrent character, pain caused by migraine, postoperative pain, phantom limb pain, inflammatory pain, neuropathic pain, chronic headache, central pain, pain related to diabetic neuropathy, to postherpetic neuralgia, or to peripheral nerve injury, bulimia, post-traumatic syndrome, social phobia, sleeping disorders, pseudodementia, Ganser's syndrome, pre-menstrual syndrome, late luteal phase syndrome, chronic fatigue syndrome, mutism, trichotillomania, jet-lag, arrhythmias, smooth muscle contractions, angina pectoris, premature labour, diarrhoea, asthma, tardive dyskinesia, hyperkinesia, premature ejaculation, erectile difficulty, hypertension, inflammatory disorders, inflammatory skin disorders, acne, rosacea, Crohn's disease, inflammatory bowel disease, ulcerative colitis, diarrhoea, or abuse liability and withdrawal symptoms caused by termination of use of addictive substances, including nicotine containing products, in particular tobacco, opioids, in particular heroin, cocaine and morphine, benzodiazepines and benzodiazepine-like drugs, and alcohol.

In a more preferred embodiment the compounds of the invention are used for the treatment, prevention or alleviation of pain, mild or moderate or severe pain, pain of acute, chronic or recurrent character, pain caused by migraine, postoperative pain, phantom limb pain, inflammatory pain, neuropathic pain, chronic headache, central pain, pain related to diabetic neuropathy, to postherpetic neuralgia, or to peripheral nerve injury.

In another more preferred embodiment the compounds of the invention are used for the treatment, prevention or alleviation of smooth muscle contractions, convulsive disorders, angina pectoris, premature labour, convulsions, diarrhoea, asthma, epilepsy, tardive dyskinesia, hyperkinesia, premature ejaculation, or erectile difficulty.

In a third more preferred embodiment the compounds of the invention are used for the treatment, prevention or alleviation of a neurodegenerative disorder, transient anoxia, or induced neuro-degeneration.

In a fourth more preferred embodiment the compounds of the invention are used for the treatment, prevention or alleviation of an inflammatory disorder, inflammatory skin disorder, acne, rosacea, Crohn's disease, inflammatory bowel disease, ulcerative colitis, or diarrhoea.

In a fifth more preferred embodiment the compounds of the invention are used for the treatment, prevention or alleviation of diabetic neuropathy, schizophrenia, cognitive or attention deficits related to schizophrenia, or depression.

In a sixth more preferred embodiment the compounds of the invention are used for the treatment, prevention or alleviation of pain, in particular neuropathic pain, diabetic neuropathy, schizophrenia and cognitive or attentional deficits related to schizophrenia, depression, and for assisting in obtaining smoking cessation.

In a seventh more preferred embodiment the compounds of the invention are used the treatment of abuse liability and withdrawal symptoms caused by termination of use of addictive substances, in particular nicotine containing products, in particular tobacco, opioids, in particular heroin, cocaine and morphine, cannabis, benzodiazepines, benzodiazepine-like drugs, and alcohol.

In an eight more preferred embodiment the compounds of the invention are used for the treatment of anxiety, cognitive disorders, learning deficit, memory deficits and dysfunction, Down's syndrome, Alzheimer's disease, attention deficit, attention deficit hyperactivity disorder (ADHD), Parkinson's disease, Huntington's disease, Amyotrophic Lateral Sclerosis, Gilles de la Tourette's syndrome, psychosis, depression, mania, manic depression, schizophrenia, obsessive compulsive disorders (OCD), panic disorders, eating disorders, in particular anorexia nervosa, bulimia and obesity, narcolepsy, nociception, AIDS-dementia, senile dementia, peripheral neuropathy, autism, dyslexia, tardive dyskinesia, hyperkinesia, epilepsy, bulimia, post-traumatic syndrome, social phobia, sleeping disorders, pseudodementia, Ganser's syndrome, pre-menstrual syndrome, late luteal phase syndrome, chronic fatigue syndrome, mutism, trichotillomania, and jet-lag.

In a ninth more preferred embodiment the compounds of the invention are used for the treatment of cognitive disorders, psychosis, schizophrenia and/or depression.

In a further more preferred embodiment the compounds of the invention are used for the treatment of endocrine disorders, in particular thyrotoxicosis, pheochromocytoma, hypertension and arrhythmias.

In a further more preferred embodiment the compounds of the invention are used for the treatment of inflammatory diseases, disorders, or conditions, including inflammatory skin disorders, in particular acne and rosacea, Crohn's disease, inflammatory bowel disease, ulcerative colitis, and diarrhoea.

Finally, in a most preferred embodiment, the compounds of the invention may be useful for the treatment of cognition, schizophrenia, attention deficit hyperactivity disorder (ADHD) and neuropathic pain.

In this context “treatment” covers treatment, prevention, prophylactics and alleviation of abuse liability and withdrawal symptoms and abstinence as well as treatment resulting in a voluntary diminished intake of the addictive substance.

In another aspect, the compounds of the invention are used as diagnostic agents, e.g. for the identification and localisation of nicotinic receptors in various tissues.

### Pharmaceutical Compositions

In another aspect the invention provides novel pharmaceutical compositions comprising a therapeutically effective amount of a tetrazole derivative of the invention.

In a preferred embodiment the pharmaceutical composition of the invention comprises a therapeutically effective amount of the tetrazole derivative of the invention, any of its isomers or any mixture of isomers, or a pharmaceutically acceptable addition salt thereof.

While a compound of the invention for use in therapy may be administered in the form of the raw compound, it is preferred to introduce the active ingredient, optionally in the form of a physiologically acceptable salt, in a pharmaceutical composition together with one or more adjuvants, excipients, carriers, buffers, diluents, and/or other customary pharmaceutical auxiliaries.

In a preferred embodiment, the invention provides pharmaceutical compositions comprising the tetrazole derivative of the invention, or a pharmaceutically acceptable salt or derivative thereof, together with one or more pharmaceutically acceptable carriers therefore, and, optionally, other therapeutic and/or prophylactic ingredients, know and used in the art. The carrier(s) must be “acceptable” in the sense of being compatible with the other ingredients of the formulation and not harmful to the recipient thereof.

The pharmaceutical composition of the invention may be administered by any convenient route, which suits the desired therapy. Preferred routes of administration include oral administration, in particular in tablet, in capsule, in dragé, in powder, or in liquid form, and parenteral administration, in particular cutaneous, subcutaneous, intramuscular, or intravenous injection. The pharmaceutical composition of the invention can be manufactured by the skilled person by use of standard methods and conventional techniques appropriate to the desired formulation. When desired, compositions adapted to give sustained release of the active ingredient may be employed.

Pharmaceutical compositions of the invention may be those suitable for oral, rectal, bronchial, nasal, pulmonal, topical (including buccal and sub-lingual), transdermal, vaginal or parenteral (including cutaneous, subcutaneous, intramuscular, intraperitoneal, intravenous, intraarterial, intracerebral, intraocular injection or infusion) administration, or those in a form suitable for administration by inhalation or insufflation, including powders and liquid aerosol administration, or by sustained release systems. Suitable examples of sustained release systems include semipermeable matrices of solid hydrophobic polymers containing the compound of the invention, which matrices may be in form of shaped articles, e.g. films or microcapsules.

The chemical compound of the invention, together with a conventional adjuvant, carrier, or diluent, may thus be placed into the form of pharmaceutical compositions and unit dosages thereof. Such forms include solids, and in particular tablets, filled capsules, powder and pellet forms, and liquids, in particular aqueous or non-aqueous solutions, suspensions, emulsions, elixirs, and capsules filled with the same, all for oral use, suppositories for rectal administration, and sterile injectable solutions for parenteral use. Such pharmaceutical compositions and unit dosage forms thereof may comprise conventional ingredients in conventional proportions, with or without additional active compounds or principles, and such unit dosage forms may contain any suitable effective amount of the active ingredient commensurate with the intended daily dosage range to be employed.

The chemical compound of the present invention can be administered in a wide variety of oral and parenteral dosage forms. It will be obvious to those skilled in the art that the following dosage forms may comprise, as the active component, either a chemical compound of the invention or a pharmaceutically acceptable salt of a chemical compound of the invention.

For preparing pharmaceutical compositions from a chemical compound of the present invention, pharmaceutically acceptable carriers can be either solid or liquid. Solid form preparations include powders, tablets, pills, capsules, cachets, suppositories, and dispersible granules. A solid carrier can be one or more substances which may also act as diluents, flavouring agents, solubilizers, lubricants, suspending agents, binders, preservatives, tablet disintegrating agents, or an encapsulating material.

In powders, the carrier is a finely divided solid, which is in a mixture with the finely divided active component.

In tablets, the active component is mixed with the carrier having the necessary binding capacity in suitable proportions and compacted in the shape and size desired.

The powders and tablets preferably contain from five or ten to about seventy percent of the active compound. Suitable carriers are magnesium carbonate, magnesium stearate, talc, sugar, lactose, pectin, dextrin, starch, gelatin, tragacanth, methylcellulose, sodium carboxymethylcellulose, a low melting wax, cocoa butter, and the like. The term “preparation” is intended to include the formulation of the active compound with encapsulating material as carrier providing a capsule in which the active component, with or without carriers, is surrounded by a carrier, which is thus in association with it. Similarly, cachets and lozenges are included. Tablets, powders, capsules, pills, cachets, and lozenges can be used as solid forms suitable for oral administration.

For preparing suppositories, a low melting wax, such as a mixture of fatty acid glyceride or cocoa butter, is first melted and the active component is dispersed homogeneously therein, as by stirring. The molten homogenous mixture is then poured into convenient sized moulds, allowed to cool, and thereby to solidify.

Compositions suitable for vaginal administration may be presented as pessaries, tampons, creams, gels, pastes, foams or sprays containing in addition to the active ingredient such carriers as are known in the art to be appropriate.

Liquid preparations include solutions, suspensions, and emulsions, for example, water or water-propylene glycol solutions. For example, parenteral injection liquid preparations can be formulated as solutions in aqueous polyethylene glycol solution.

The chemical compound according to the present invention may thus be formulated for parenteral administration (e.g. by injection, for example bolus injection or continuous infusion) and may be presented in unit dose form in ampoules, pre-filled syringes, small volume infusion or in multi-dose containers with an added preservative. The compositions may take such forms as suspensions, solutions, or emulsions in oily or aqueous vehicles, and may contain formulation agents such as suspending, stabilising and/or dispersing agents. Alternatively, the active ingredient may be in powder form, obtained by aseptic isolation of sterile solid or by lyophilization from solution, for constitution with a suitable vehicle, e.g. sterile, pyrogen-free water, before use.

Aqueous solutions suitable for oral use can be prepared by dissolving the active component in water and adding suitable colorants, flavours, stabilising and thickening agents, as desired.

Aqueous suspensions suitable for oral use can be made by dispersing the finely divided active component in water with viscous material, such as natural or synthetic gums, resins, methylcellulose, sodium carboxymethylcellulose, or other well known suspending agents.

Also included are solid form preparations, intended for conversion shortly before use to liquid form preparations for oral administration. Such liquid forms include solutions, suspensions, and emulsions. In addition to the active component such preparations may comprise colorants, flavours, stabilisers, buffers, artificial and natural sweeteners, dispersants, thickeners, solubilizing agents, and the like.

For topical administration to the epidermis the chemical compound of the invention may be formulated as ointments, creams or lotions, or as a transdermal patch. Ointments and creams may, for example, be formulated with an aqueous or oily base with the addition of suitable thickening and/or gelling agents. Lotions may be formulated with an aqueous or oily base and will in general also contain one or more emulsifying agents, stabilising agents, dispersing agents, suspending agents, thickening agents, or colouring agents.

Compositions suitable for topical administration in the mouth include lozenges comprising the active agent in a flavoured base, usually sucrose and acacia or tragacanth; pastilles comprising the active ingredient in an inert base such as gelatin and glycerine or sucrose and acacia; and mouthwashes comprising the active ingredient in a suitable liquid carrier.

Solutions or suspensions are applied directly to the nasal cavity by conventional means, for example with a dropper, pipette or spray. The compositions may be provided in single or multi-dose form.

Administration to the respiratory tract may also be achieved by means of an aerosol formulation in which the active ingredient is provided in a pressurised pack with a suitable propellant such as a chlorofluorocarbon (CFC) for example dichlorodifluoromethane, trichlorofluoromethane, or dichlorotetrafluoroethane, carbon dioxide, or other suitable gas. The aerosol may conveniently also contain a surfactant such as lecithin. The dose of drug may be controlled by provision of a metered valve.

Alternatively the active ingredients may be provided in the form of a dry powder, for example a powder mix of the compound in a suitable powder base such as lactose, starch, starch derivatives such as hydroxypropylmethyl cellulose and polyvinylpyrrolidone (PVP). Conveniently the powder carrier will form a gel in the nasal cavity. The powder composition may be presented in unit dose form for example in capsules or cartridges of, e.g., gelatin, or blister packs from which the powder may be administered by means of an inhaler.

In compositions intended for administration to the respiratory tract, including intranasal compositions, the compound will generally have a small particle size for example of the order of 5 microns or less. Such a particle size may be obtained by means known in the art, for example by micronization.

When desired, compositions adapted to give sustained release of the active ingredient may be employed.

The pharmaceutical preparations are preferably in unit dosage forms. In such form, the preparation is subdivided into unit doses containing appropriate quantities of the active component. The unit dosage form can be a packaged preparation, the package containing discrete quantities of preparation, such as packaged tablets, capsules, and powders in vials or ampoules. Also, the unit dosage form can be a capsule, tablet, cachet, or lozenge itself, or it can be the appropriate number of any of these in packaged form.

Tablets or capsules for oral administration and liquids for intravenous administration and continuous infusion are preferred compositions.

In a preferred embodiment, when the pharmaceutical composition of the invention is intended for treating patients with abuse liability and withdrawal symptoms caused by nicotine addiction, formulations such as gums, patches, sprays, inhalers, aerosols, etc., are contemplated.

Further details on techniques for formulation and administration may be found in the latest edition of Remington's Pharmaceutical Sciences (Maack Publishing Co., Easton, Pa.).

A therapeutically effective dose refers to that amount of active ingredient, which ameliorates the symptoms or condition. Therapeutic efficacy and toxicity, e.g. ED₅₀ and LD₅₀, may be determined by standard pharmacological procedures in cell cultures or experimental animals. The dose ratio between therapeutic and toxic effects is the therapeutic index and may be expressed by the ratio LD₅₀/ED₅₀. Pharmaceutical compositions exhibiting large therapeutic indexes are preferred.

The dose administered must of course be carefully adjusted to the age, weight and condition of the individual being treated, as well as the route of administration, dosage form and regimen, and the result desired, and the exact dosage should of course be determined by the practitioner.

The actual dosage depends on the nature and severity of the disease being treated, and is within the discretion of the physician, and may be varied by titration of the dosage to the particular circumstances of this invention to produce the desired therapeutic effect. However, it is presently contemplated that pharmaceutical compositions containing of from about 0.1 to about 500 mg of active ingredient per individual dose, preferably of from about 1 to about 100 mg, most preferred of from about 1 to about 10 mg, are suitable for therapeutic treatments.

The active ingredient may be administered in one or several doses per day. A satisfactory result can, in certain instances, be obtained at a dosage as low as 0.1 μg/kg i.v. and 1 μg/kg p.o. The upper limit of the dosage range is presently considered to be about 10 mg/kg i.v. and 100 mg/kg p.o. Preferred ranges are from about 0.1 μg/kg to about 10 mg/kg/day i.v., and from about 1 μg/kg to about 100 mg/kg/day p.o.

### Methods of Therapy

The tetrazole derivatives of the present invention are valuable nicotinic receptor modulators, and therefore useful for the treatment of a range of ailments involving cholinergic dysfunction as well as a range of disorders responsive to the action of nAChR modulators.

In another aspect the invention provides a method for the treatment, prevention or alleviation of a disease or a disorder or a condition of a living animal body, including a human, which disease, disorder or condition is responsive to modulation of cholinergic receptors, and which method comprises administering to such a living animal body, including a human, in need thereof an effective amount of a tetrazole derivative of the invention.

In the context of this invention the term “treatment” covers treatment, prevention, prophylaxis or alleviation, and the term “disease” covers illnesses, diseases, disorders and conditions related to the disease in question.

The preferred indications contemplated according to the invention are those stated above.

It is at present contemplated that suitable dosage ranges are 0.1 to 1000 milligrams daily, 10-500 milligrams daily, and especially 30-100 milligrams daily, dependent as usual upon the exact mode of administration, form in which administered, the indication toward which the administration is directed, the subject involved and the body weight of the subject involved, and further the preference and experience of the physician or veterinarian in charge.

A satisfactory result can, in certain instances, be obtained at a dosage as low as 0.005 mg/kg i.v. and 0.01 mg/kg p.o. The upper limit of the dosage range is about 10 mg/kg i.v. and 100 mg/kg p.o. Preferred ranges are from about 0.001 to about 1 mg/kg i.v. and from about 0.1 to about 10 mg/kg p.o.

### EXAMPLES

The invention is further illustrated with reference to the following examples, which are not intended to be in any way limiting to the scope of the invention as claimed.

### Example 1

### Preparatory Example

All reactions involving air sensitive reagents or intermediates were performed under nitrogen and in anhydrous solvents. Magnesium sulfate was used as drying agent in the workup-procedures and solvents were evaporated under reduced pressure.

### Method A

### 3-Fluoro-5-[5-(3-pyridyl)tetrazol-2-yl]pyridine Free Base (Compound A1)

A mixture of 4-methyl-N-(3-pyridylmethyleneamino) benzene sulfonamide (1.0 g, 3.63 mmol), 3-amino-5-fluoropyridine (0.41 g, 3.63 mmol), pyridine (4 ml) and THF (8 ml) was stirred under nitrogen. Butylnitrite (0.51 g, 4.72 mmol) was added dropwise and was stirred at room-temperature for 7 days. The solvent was evaporated, water was added and the mixture was extracted with dichloromethane. The mixture was purified by silica column chromatography, using a mixture of petroleum and ethylacetate as eluent. Yield 360 mg (41%). LC-ESI-HRMS of [M+H]+ shows 243.07825 Da. Calc. 243.078902 Da, dev. −2.7 ppm.

### Method B

### 4-Methyl-N-(3-pyridylmethyleneamino)benzenesulfonamide (Intermediate Compound)

A mixture of p-toluenesulfonhydrazide (10.4 g, 56.0 mmol), ethanol (140 ml, 96%) and aqueous hydrochloric acid (1.5 ml, 1 M) was heated to reflux. Another mixture of 3-pyridinecarboxaldehyde (6.0 g, 56.0 mmol) and ethanol (60 ml, 96%) was added dropwise, followed by stirring at reflux at 30 min. The heating was removed and the mixture was allowed to stir at room-temperature over night. The solid product was isolated by filtration and washed with water. Yield 11.59 g (75%).

### 3-[2-(3-Pyridyl)tetrazol-5-yl]pyridine fumarate (Compound B1)

Was prepared according to Methods A and B. LC-ESI-HRMS of [M+H]+ shows 225.08818 Da. Calc. 225.088324 Da, dev. −0.6 ppm.

### 3-Bromo-5-[5-(3-pyridyl)tetrazol-2-yl]pyridine Free Base (Compound B2)

Was prepared according to Methods A and B. LC-ESI-HRMS of [M+H]+ shows 302.99839 Da. Calc. 302.998837 Da, dev. −1.5 ppm.

### 3-Methoxy-5-[5-(3-pyridyl)tetrazol-2-yl]pyridine Free Base (Compound B3)

Was prepared according to Methods A and B. LC-ESI-HRMS of [M+H]+ shows 255.0985 Da. Calc. 255.098889 Da, dev. −1.5 ppm.

### 2-Methyl-5-[2-(3-pyridyl)tetrazol-5-yl]pyridine Free Base (Compound B4)

Was prepared according to Methods A and B. LC-ESI-HRMS of [M+H]+ shows 239.10336 Da. Calc. 239.103974 Da, dev. −2.6 ppm.

### 2-Methyl-5-[5-(3-pyridyl)tetrazol-2-yl]pyridine Free Base (Compound B5)

Was prepared according to Methods A and B. LC-ESI-HRMS of [M+H]+ shows 239.10335 Da. Calc. 239.103974 Da, dev. −2.6 ppm.

### 3-Methyl-5-[5-(3-pyridyl)tetrazol-2-yl]pyridine Free Base (Compound B6)

Was prepared according to Methods A and B. LC-ESI-HRMS of [M+H]+ shows 239.10327 Da. Calc. 239.103974 Da, dev. −2.9 ppm.

### 2-Methyl-3-[5-(3-pyridyl)tetrazol-2-yl]pyridine Free Base (Compound B7)

Was prepared according to Methods A and B. LC-ESI-HRMS of [M+H]+ shows 239.10321 Da. Calc. 239.103974 Da, dev. −3.2 ppm.

### 2-Methyl-3-[2-(3-pyridyl)tetrazol-5-yl]pyridine Free Base (Compound B8)

Was prepared according to Methods A and B. LC-ESI-HRMS of [M+H]+ shows 239.10336 Da. Calc. 239.103974 Da, dev. −2.6 ppm.

### 2-Fluoro-5-[5-(3-pyridyl)tetrazol-2-yl]pyridine Free Base (Compound B9)

Was prepared according to Methods A and B. LC-ESI-HRMS of [M+H]+ shows 243.07861 Da. Calc. 243.078902 Da, dev. −1.2 ppm.

### 3-Fluoro-5-[2-(5-fluoro-3-pyridyl)tetrazol-5-yl]pyridine Free Base (Compound B10)

Was prepared according to Methods A and B. LC-ESI-HRMS of [M+H]+ shows 261.06945 Da. Calc. 261.06948 Da, dev. −0.1 ppm.

### 3-Fluoro-5-[2-(6-fluoro-3-pyridyl)tetrazol-5-yl]pyridine Free Base (Compound B11)

Was prepared according to Methods A and B. LC-ESI-HRMS of [M+H]+ shows 261.06914 Da. Calc. 261.06948 Da, dev. −1.3 ppm.

### 2-Fluoro-5-[2-(5-fluoro-3-pyridyl)tetrazol-5-yl]pyridine Free Base (Compound B12)

Was prepared according to Methods A and B. LC-ESI-HRMS of [M+H]+ shows 261.06901 Da. Calc. 261.06948 Da, dev. −1.8 ppm.

### 2-Chloro-5-[2-(3-pyridyl)tetrazol-5-yl]pyridine Free Base (Compound B13)

Was prepared according to Methods A and B. LC-ESI-HRMS of [M+H]+ shows 259.04899 Da. Calc. 259.049352 Da, dev. −1.4 ppm.

### 2-Fluoro-5-[2-(3-pyridyl)tetrazol-5-yl]pyridine Free Base (Compound B14)

Was prepared according to Methods A and B. LC-ESI-HRMS of [M+H]+ shows 243.07861 Da. Calc. 243.078902 Da, dev. −1.2 ppm.

### 3-Chloro-5-[2-(3-pyridyl)tetrazol-5-yl]pyridine Free Base (Compound B15)

Was prepared according to Methods A and B. LC-ESI-HRMS of [M+H]+ shows 259.04925 Da. Calc. 259.049352 Da, dev. −0.4 ppm.

### 3-Fluoro-5-[2-(3-pyridyl)tetrazol-5-yl]pyridine Free Base (Compound B16)

Was prepared according to Methods A and B. LC-ESI-HRMS of [M+H]+ shows 243.07867 Da. Calc. 243.078902 Da, dev. −1 ppm.

### 2-chloro-5-[5-(3-pyridyl)tetrazol-2-yl]pyridine Free Base (Compound B17)

Was prepared according to Methods A and B. LC-ESI-HRMS of [M+H]+ shows 259.04945 Da. Calc. 259.049352 Da, dev. 0.4 ppm.

### 3-chloro-5-[2-(6-fluoro-3-pyridyl)tetrazol-5-yl]pyridine Free Base (Compound B18)

Was prepared according to Methods A and B. LC-ESI-HRMS of [M+H]+ shows 277.03924 Da. Calc. 277.03993 Da, dev. −2.5 ppm.

### 2-chloro-5-[5-(5-fluoro-3-pyridyl)tetrazol-2-yl]pyridine Free Base (Compound B19)

Was prepared according to Methods A and B. LC-ESI-HRMS of [M+H]+ shows 277.03932 Da. Calc. 277.03993 Da, dev. −2.2 ppm.

### 2-chloro-5-[2-(5-fluoro-3-pyridyl)tetrazol-5-yl]pyridine Free Base (Compound B20)

Was prepared according to Methods A and B. LC-ESI-HRMS of [M+H]+ shows 277.03955 Da. Calc. 277.03993 Da, dev. −1.4 ppm.

### 3-chloro-5-[5-(3-pyridyl)tetrazol-2-yl]pyridine Free Base (Compound B21)

Was prepared according to Methods A and B. LC-ESI-HRMS of [M+H]+ shows 259.04947 Da. Calc. 259.049352 Da, dev. 0.5 ppm.

### 3-chloro-5-[2-(5-fluoro-3-pyridyl)tetrazol-5-yl]pyridine Free Base (Compound B22)

Was prepared according to Methods A and B. LC-ESI-HRMS of [M+H]+ shows 277.03996 Da. Calc. 277.03993 Da, dev. 0.1 ppm.

### 3-bromo-5-[2-(3-pyridyl)tetrazol-5-yl]pyridine Free Base (Compound B23)

Was prepared according to Methods A and B. LC-ESI-HRMS of [M+H]+ shows 302.99867 Da. Calc. 302.998837 Da, dev. −0.6 ppm.

### 2-fluoro-3-[2-(6-fluoro-3-pyridyl)tetrazol-5-yl]pyridine Free Base (Compound B24)

Was prepared according to Methods A and B. LC-ESI-HRMS of [M+H]+ shows 261.06952 Da. Calc. 261.06948 Da, dev. 0.2 ppm.

### 2-fluoro-5-[5-(5-iodo-3-pyridyl)tetrazol-2-yl]pyridine Free Base (Compound B25)

Was prepared according to Methods A and B. LC-ESI-HRMS of [M+H]+ shows 368.9759 Da. Calc. 368.975577 Da, dev. 0.9 ppm.

### 2-chloro-5-[5-(5-chloro-3-pyridyl)tetrazol-2-yl]pyridine Free Base (Compound B26)

Was prepared according to Methods A and B. LC-ESI-HRMS of [M+H]+ shows 368.9759 Da. Calc. 368.975577 Da, dev. 0.9 ppm.

### Example 2

### Biological Activity

Characterization of hα4β2 Positive Allosteric Modulators Using FLIPR

This experiment shows the modulating activity of compounds of the invention by the ability to positively modulate the response induced by a range of concentrations of nicotine (0.1 nM-100 mM) in a HEK-293 cell line stably expressing the human nicotinic acetylcholine receptor subtype a4b2. The modulator activity is determined as a fluorescence-based assay using a Fluorometric Imaging Plate Reader (FLIPR) as described below in more detail.

HEK-hα4β2 cells were seeded on poly-D-lysin-coated 384 well microtiter plates and allowed to proliferate for 24 h. Dye loading was performed by incubating cells with 2 μM fluo-4/AM for 1.5 h at room temperature. Dye not taken up by the cells was removed by aspiration, followed by three washing cycles with NMDG Ringer buffer (containing in mM: 140 NMDG, 5 KCl, 1 MgCl₂, 10 CaCl₂, 10 HEPES, pH 7.4). The microtiter plates were then placed in the FLIPR and subjected to test solutions.

Full concentration-response curves of nicotine were generated in the absence and presence test compound. Background-subtracted nicotine-mediated calcium responses were determined and normalized to maximal nicotine control responses. Nicotine potencies (expressed as pEC₅₀) were calculated and the difference between the functional potency in the absence and presence of test compound was determined (Δ pEC₅₀).

The modulatory activity of compounds of the invention determined as ΔpEC₅₀ values in the presence of 10 μM of test compound are presented in Table 1 below.

TABLE 1
FLIPR nAChR α4β2 positive allosteric modulator activity

| Compound | ΔpEC₅₀ |
|---|---|
| A1 | 1.5 |
| B1 | 1.8 |
| B2 | 0.55 |
| B3 | −0.16 |
| B4 | 1.0 |
| B5 | 1.7 |
| B6 | 0.16 |
| B7 | 0.11 |
| B8 | −0.29 |
| B9 | 1.9 |
| B10 | 1.3 |
| B11 | 1.4 |
| B12 | 1.6 |
| B13 | 1.8 |
| B14 | 1.5 |
| B15 | 1.7 |
| B16 | 1.7 |
| B17 | 2.0 |
| B18 | 1.5 |
| B19 | 1.6 |
| B20 | 0.74 |
| B21 | 0.8 |
| B22 | 1.1 |
| B23 | 1.5 |
| B24 | 1.6 |
| B25 | 0.58 |
| B26 | 0.95 |

1-9. (canceled)

10. A tetrazole derivative represented by Formula I: 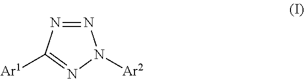
a stereoisomer thereof or a mixture of its stereoisomers, or a pharmaceutically acceptable addition salt thereof, wherein both of Ar¹ and Ar² represent pyridinyl, optionally substituted one or two times with substituents selected from alkyl, alkoxy and halo.

11. The tetrazole derivative of claim 10, a stereoisomer thereof or a mixture of its stereoisomers, or a pharmaceutically acceptable addition salt thereof, wherein
Ar¹ represents pyridinyl, and in particular 3-pyridinyl, optionally substituted with alkyl or halo; and
Ar² represents pyridinyl, and in particular 3-pyridinyl, optionally substituted with alkyl, alkoxy or halo.

12. The tetrazole derivative of claim 10, a stereoisomer thereof or a mixture of its stereoisomers, or a pharmaceutically acceptable addition salt thereof, wherein
Ar¹ represents 3-pyridinyl, optionally substituted with alkyl or halo; and
Ar² represents 3-pyridinyl, optionally substituted with alkyl, alkoxy or halo;
with the proviso that at least one of Ar¹ and Ar² represents a substituted 3-pyridinyl; and
with the further proviso that Ar² does not represent 6-chloro-pyridin-3-yl if Ar¹ represents pyridin-3-yl or 2-chloro-pyridin-3-yl.

13. The tetrazole derivative compound of claim 10, which is
3-Fluoro-5-[5-(3-pyridyl)tetrazol-2-yl]pyridine;
3-[2-(3-Pyridyl)tetrazol-5-yl]pyridine fumarate;
3-Bromo-5-[5-(3-pyridyl)tetrazol-2-yl]pyridine;
3-Methoxy-5-[5-(3-pyridyl)tetrazol-2-yl]pyridine;
2-Methyl-5-[2-(3-pyridyl)tetrazol-5-yl]pyridine;
2-Methyl-5-[5-(3-pyridyl)tetrazol-2-yl]pyridine;
3-Methyl-5-[5-(3-pyridyl)tetrazol-2-yl]pyridine;
2-Methyl-3-[5-(3-pyridyl)tetrazol-2-yl]pyridine;
2-Methyl-3-[2-(3-pyridyl)tetrazol-5-yl]pyridine;
2-Fluoro-5-[5-(3-pyridyl)tetrazol-2-yl]pyridine;
3-Fluoro-5-[2-(5-fluoro-3-pyridyl)tetrazol-5-yl]pyridine;
3-Fluoro-5-[2-(6-fluoro-3-pyridyl)tetrazol-5-yl]pyridine;
2-Fluoro-5-[2-(5-fluoro-3-pyridyl)tetrazol-5-yl]pyridine;
2-Chloro-5-[2-(3-pyridyl)tetrazol-5-yl]pyridine;
2-Fluoro-5-[2-(3-pyridyl)tetrazol-5-yl]pyridine;
3-Chloro-5-[2-(3-pyridyl)tetrazol-5-yl]pyridine;
3-Fluoro-5-[2-(3-pyridyl)tetrazol-5-yl]pyridine;
2-Chloro-5-[5-(3-pyridyl)tetrazol-2-yl]pyridine;
3-Chloro-5-[2-(6-fluoro-3-pyridyl)tetrazol-5-yl]pyridine;
2-Chloro-5-[5-(5-fluoro-3-pyridyl)tetrazol-2-yl]pyridine;
2-Chloro-5-[2-(5-fluoro-3-pyridyl)tetrazol-5-yl]pyridine;
3-Chloro-5-[5-(3-pyridyl)tetrazol-2-yl]pyridine;
3-Chloro-5-[2-(5-fluoro-3-pyridyl)tetrazol-5-yl]pyridine;
3-Bromo-5-[2-(3-pyridyl)tetrazol-5-yl]pyridine;
2-Fluoro-3-[2-(6-fluoro-3-pyridyl)tetrazol-5-yl]pyridine;
2-Fluoro-5-[5-(5-iodo-3-pyridyl)tetrazol-2-yl]pyridine; or
2-Chloro-5-[5-(5-chloro-3-pyridyl)tetrazol-2-yl]pyridine;
a stereoisomer thereof or a mixture of its stereoisomers, or a pharmaceutically acceptable addition salt thereof.

14. A pharmaceutical composition comprising a therapeutically effective amount of a tetrazole derivative of claim 10, a stereoisomer thereof or a mixture of its stereoisomers, or a pharmaceutically acceptable addition salt thereof, together with at least one pharmaceutically acceptable carrier or diluent.

15. A method of treatment, prevention or alleviation of a disease or a disorder or a condition of a living animal body, including a human, which disorder, disease or condition is responsive to modulation of cholinergic receptors, which method comprises the step of administering to such a living animal body in need thereof a therapeutically effective amount of a tetrazole derivative of claim 10, a stereoisomer thereof or a mixture of its stereoisomers, or a pharmaceutically acceptable addition salt thereof.

16. The method according to claim 15, wherein the disease, disorder or condition is a cognitive disorder, learning deficit, memory deficits and dysfunction, Down's syndrome, Alzheimer's disease, attention deficit, attention deficit hyperactivity disorder (ADHD), Tourette's syndrome, psychosis, depression, bipolar disorder, mania, manic depression, schizophrenia, cognitive or attention deficits related to schizophrenia, obsessive compulsive disorders (OCD), panic disorders, eating disorders such as anorexia nervosa, bulimia and obesity, narcolepsy, nociception, AIDS-dementia, senile dementia, autism, Parkinson's disease, Huntington's disease, amyotrophic lateral sclerosis (ALS), anxiety, non-OCD anxiety disorders, convulsive disorders, convulsions, epilepsy, neurodegenerative disorders, transient anoxia, induced neuro-degeneration, neuropathy, diabetic neuropathy, peripheral dyslexia, tardive dyskinesia, hyperkinesia, pain, mild pain, moderate or severe pain, pain of acute, chronic or recurrent character, pain caused by migraine, postoperative pain, phantom limb pain, inflammatory pain, neuropathic pain, chronic headache, central pain, pain related to diabetic neuropathy, to postherpetic neuralgia, or to peripheral nerve injury, post-traumatic syndrome, social phobia, sleeping disorders, pseudodementia, Ganser's syndrome, pre-menstrual syndrome, late luteal phase syndrome, chronic fatigue syndrome, mutism, trichotillomania, jet-lag, arrhythmias, smooth muscle contractions, angina pectoris, premature labour, diarrhoea, asthma, tardive dyskinesia, hyperkinesia, premature ejaculation, erectile difficulty, hypertension, inflammatory disorders, inflammatory skin disorders, acne, rosacea, Crohn's disease, inflammatory bowel disease, ulcerative colitis, diarrhoea, or abuse liability and withdrawal symptoms caused by termination of use of addictive substances, including nicotine containing products such as tobacco, opioids such as heroin, cocaine and morphine, cannabis, benzodiazepines and benzodiazepine-like drugs, and alcohol.
